(19) United States
(12) Patent Application Publication
(10) Pub. No.: US 2026/0021223 A1
(43) Pub. Date: Jan. 22, 2026

(54) HYDROGEL FILLERS FOR CONFORMABLE FILLABLE MEDICAL BALLOONS

(71) Applicant: Boston Scientific Scimed, Inc., Maple Grove, MN (US)

(72) Inventors: Nicolas Ball-Jones, Minneapolis, MN (US); Rick William Dorn, Plymouth, MN (US); Cristian Parisi, Amherst, MA (US); Joseph Thomas Delaney, Jr., Minneapolis, MN (US)

(73) Assignee: Boston Scientific Scimed, Inc., Maple Grove, MN (US)

(21) Appl. No.: 19/270,195

(22) Filed: Jul. 15, 2025

Related U.S. Application Data
(60) Provisional application No. 63/673,090, filed on Jul. 18, 2024.

(51) Int. Cl. A61L 27/52; A61L 27/16; A61L 27/58

(52) U.S. Cl. CPC A61L 27/52; A61L 27/16; A61L 27/58

(57) ABSTRACT

In various aspects, the present disclosure pertains to balloon implantation kits comprising: (a) a balloon configured to be implanted in a mammalian body and (b) a reservoir containing a filler material that is configured to be injected into the balloon, the filler material selected from a hydrogel filler material comprising crosslinked hydrophilic polymer chains or hydrogel precursors that form a hydrogel filler material in the balloon when combined.

### CROSS-REFERENCE TO RELATED APPLICATIONS

This application claims the benefit of U.S. Provisional Patent Application Ser. No. 63/673,090 filed on Jul. 18, 2024, the disclosure of which is incorporated herein by reference.

### FIELD

The present disclosure pertains to hydrogel fillers for conformable fillable balloons that are configured to be implanted in a mammalian body for various medical uses.

### BACKGROUND

Various solutions presently exist for spacing, lifting, and embolic applications. However, injectable materials, including in-situ crosslinking materials and shear-thinning materials, can have some potential inconveniences that include asymmetric localized deployment of the implanted material, potential for off-target embolization due to delayed reaction or migration of the implanted material, and complexity associated with removal of material if complete removal of the material is desired.

The present disclosure addresses these and other inconveniences by employing conformable, fillable balloons to confine injectable materials at the implantation site.

### SUMMARY

The present disclosure is directed to filler materials for conformable, fillable balloons that are suitable for implantation in a mammalian body.

In some aspects, the present disclosure pertains to balloon implantation kits that comprise: (a) a balloon configured to be implanted in a mammalian body and (b) a reservoir containing a filler material that is configured to be injected into the balloon, the filler material selected from a hydrogel filler material comprising crosslinked hydrophilic polymer chains or hydrogel precursors that form a hydrogel filler material in the balloon when combined.

In some embodiments, which can be used in conjunction with the above aspects, the crosslinked hydrophilic polymer chains are crosslinked by crosslinks that comprise hydrolysable linkers. For example, the hydrolysable linkers may be selected from a carbonate linker, an acid anhydride linker, an imide linker, a ketal linker, a carbamate linker, an organophosphate ester linker, a silane linker, an amide linker, a hydrozonium linker, an acylhydrozone linker, an oxime linker, an amidohydrozone linker, or a combination thereof.

In some embodiments, which can be used in conjunction with the above aspects and embodiments, the crosslinks further comprise an activating group positioned adjacent to the hydrolysable linkers, which increases a rate of hydrolysis of the hydrolysable linkers. In some cases, the activating group is selected from a hydrogen bond donor, a hydrogen bond acceptor, a lone pair donor, a lone pair acceptor, a silicon containing group, a boron containing group, a phosphonate group and a sulfonate group. In some cases, the activating group may be selected from a urea linker, a urea pendant group, a thiourea linker, a thiourea pendant group, an amine linker, an amine pendant group, an alcohol pendant group, a silicon-containing linker, a silicon-containing pendant group, a boron-containing linker, a boron-containing pendant group, a phosphonate linker, a phosphonate pendant group, a sulfonate pendant group, or a combination thereof.

In some embodiments, which can be used in conjunction with the above aspects and embodiments, the crosslinked hydrophilic polymer chains are crosslinked by crosslinks that comprise immolative linkers or by crosslinks that comprise reversible covalent linkers that incorporate reactive dimeric linkers. In some of these embodiments, the kits further comprise a cleavage composition that contains a singly reactive molecule that acts to break the crosslinks at a position of the immolative linkers within the crosslinks, or the cleavage composition contains a singly reactive molecule that acts to break the crosslinks at a position of the dimeric linkers within the crosslinks. In some of these embodiments, the kits further comprise a syringe barrel that contains the cleavage composition. In some of these embodiments, the kits may further comprise a needle, a flexible tube, or both, and the syringe barrel may be configured for coupling to the needle, the flexible tube, or both.

In some embodiments, which can be used in conjunction with the above aspects and embodiments, the hydrophilic polymer chains are crosslinked by crosslinks that comprise immolative linkers and the hydrogel cleavage composition contains a singly reactive molecule that acts to break the crosslinks at the position of the immolative linkers within the crosslinks. In some of these embodiments, the crosslinks comprise imidosydnone groups as immolative linkers and the singly reactive molecule is a strained alkyne reactive molecule. In some of these embodiments, the crosslinks comprise 1-(methyloxidoamino) cyclooctene groups as immolative linkers and the singly reactive molecule is a diboron molecule.

In some embodiments, which can be used in conjunction with the above aspects and embodiments, the hydrophilic polymer hydrogel comprises hydrophilic polymer chains that are crosslinked by crosslinks that contain reversible covalent linkers that incorporate a reactive dimeric linker, and the hydrogel cleavage composition contains a singly reactive molecule that acts to break the crosslinks at the position of the dimeric linkers within the crosslinks. In some of these embodiments, the reversible covalent linkers each comprises two thioester groups, the singly reactive molecule is a thiol molecule, and the reactive dimeric linker is a bis-thiol linker.

In some embodiments, which can be used in conjunction with the above aspects and embodiments, the hydrophilic polymer chains are selected from polyalkylene oxide chains, polyester chains, polyoxazoline chains, polydioxanone chains, polypeptide chains, polyacrylate chains, polyacrylamide chains, or combinations thereof.

In some embodiments, which can be used in conjunction with the above aspects and embodiments, the filler material is a shear-thinning hydrogel material. In some of these embodiments, the shear-thinning hydrogel material comprises fibrous protein, silicate particles and water.

In some embodiments, which can be used in conjunction with the above aspects and embodiments, the filler material is a lower critical solution temperature material that is in the form of a hydrogel at body temperature.

In some embodiments, which can be used in conjunction with the above aspects and embodiments, the filler material comprises an imaging agent.

In some embodiments, which can be used in conjunction with the above aspects and embodiments, the balloon is a biostable balloon.

In some embodiments, which can be used in conjunction with the above aspects and embodiments, the balloon is a degradable balloon.

In some embodiments, which can be used in conjunction with the above aspects and embodiments, the kits further comprise an elongate filling tube that is configured to be removably coupled to the balloon, the elongate filling tube having a lumen that is configured for fluid communication with an interior of the balloon. In some of these embodiments, the elongate filling tube is removably coupled to the balloon.

In some embodiments, which can be used in conjunction with the above aspects and embodiments, the kits further comprise a delivery sheath. In some of these embodiments, the balloon is removably disposed in the delivery sheath.

In some embodiments, which can be used in conjunction with the above aspects and embodiments, the kits comprise sterile packaging within which components of the balloon implantation kit are removably packaged in a sterile state.

Other aspects of the present disclosure pertain to methods that comprise contacting an implant, which implant comprises a conformable, fillable balloon and a hydrogel filler material disposed within the conformable, fillable balloon that comprises a hydrophilic polymer hydrogel comprising crosslinked hydrophilic polymer chains that are crosslinked by crosslinks that comprise immolative linkers or by crosslinks that contain reversible covalent linkages that incorporate reactive dimeric linkers, with a cleavage composition that contains a singly reactive molecule that acts to break the crosslinks at a position of the immolative linkers within the crosslinks or at a position of the dimeric linkers within the crosslinks, thereby accelerating breakdown of the conformable, fillable balloon.

Potential advantages of the present disclosure include one or more of the following, among others: reduced chance of unconfined localized deployment of injected material, reduced chance of asymmetric or uncontrolled distribution of injected material, reduced chance of off-target embolization, the use of a wider spectrum of injectable filler materials beyond those that are currently injected into subjects, and the ability of completely remove injected material from a subject if removal of the material is desired.

### BRIEF DESCRIPTION OF THE DRAWINGS

FIG. 1 schematically illustrates a device for filling conformable, fillable balloon with a hydrophilic polymer hydrogel, in accordance with an embodiment of the present disclosure.

FIG. 2 schematically illustrates a device for filling conformable, fillable balloon with an in-situ-forming hydrophilic polymer hydrogel, in accordance with another embodiment of the present disclosure.

FIG. 3A schematically illustrates a process for forming boc-protected 4-imidosyndonebenzoic acid chloride, in accordance with an embodiment of the present disclosure.

FIG. 3B schematically illustrates a process for forming an imidosydnone-functionalized trilysine-derived crosslinker, in accordance with an embodiment of the present disclosure.

FIG. 3C schematically illustrates a process for forming a succinimidyl-ester-terminated multi-arm polymer, in accordance with an embodiment of the present disclosure.

FIG. 3D schematically illustrates a process for forming a hydrophilic polymer hydrogel from the imidosydnone-functionalized trilysine-derived crosslinker of FIG. 3B and the succinimidyl-ester-terminated multi-arm polymer of FIG. 3C, in accordance with an embodiment of the present disclosure.

FIG. 3E schematically illustrates a process for cleaving crosslinks in the hydrophilic polymer hydrogel of FIG. 3D by contact with a strained alkyne, in accordance with an embodiment of the present disclosure.

FIG. 3F schematically illustrates a process for forming an imidosydnone-functionalized trilysine-derived crosslinker, in accordance with a further embodiment of the present disclosure.

FIG. 4A schematically illustrates a process for forming a cyclooctyne-terminated multi-arm polymer, in accordance with an embodiment of the present disclosure.

FIG. 4B schematically illustrates a process for forming an N-methylhydroxylamine-functional lysine-derived crosslinker, in accordance with an embodiment of the present disclosure.

FIG. 4C schematically illustrates a process for forming a hydrophilic polymer hydrogel from the cyclooctyne-terminated multi-arm polymer of FIG. 4A and the N-methylhydroxylamine-functional lysine-derived crosslinker of FIG. 4B, in accordance with an embodiment of the present disclosure.

FIG. 4D schematically illustrates a process for cleaving crosslinks in the hydrophilic polymer hydrogel of FIG. 4C by contact with a diboron compound, in accordance with an embodiment of the present disclosure.

FIG. 5 schematically illustrates a pre-loaded syringe that contains an injectable cleavage composition, in accordance with an embodiment of the present disclosure.

FIGS. 6A-6D schematically illustrate a method of implanting and filling a conformable, fillable balloon in a subject, in accordance with an embodiment of the present disclosure.

FIGS. 7A-7C schematically illustrate a method of breaking down a hydrophilic polymer hydrogel in a subject, in accordance with an embodiment of the present disclosure.

### DETAILED DESCRIPTION

The present disclosure is directed to hydrogel filler materials for conformable, fillable balloons that are suitable for implantation in a mammalian body, typically, a human body.

The implantable balloons are useful for a number of medical procedures including spacing, lifting, bulking, embolization and backfill procedures. Spacing procedures include soft tissue spacing procedures wherein one or more fillable balloons are placed, for example, between the rectum and the prostate and filled to prevent damage during radiation therapy or between other organs and/or tissues to protect them from potential side effects of a particular treatment, and joint spacing procedures where one or more fillable balloons are placed in the space of a joint, such as a shoulder, hip, knee or ankle joint and filled to provide mechanical support for the joint. Lifting procedures include procedures where one or more fillable balloons are placed and filled to lift a sinus membrane for sinus augmentation or others. Bulking procedures include procedures wherein one or more fillable balloons are implanted in or adjacent to a bodily sphincter, such as an anal sphincter or a urethral sphincter, to address intrinsic sphincter deficiency or others. Embolization procedures include those where one or more fillable balloons are placed in a target blood vessel and filled to block blood flow to an area of the body. Backfill procedures include procedures wherein one or more fillable balloons are implanted and filled in a left atrial appendage after the introduction of a closure device such as the Watchman® left atrial appendage closure device available from Boston Scientific Corporation, Marlborough, MA, USA, or the balloon as a closure device for the left atrial appendage with no additional support or devices.

Because the hydrogel filler materials are injected in a balloon, removal of the balloon will also result in removal of the hydrogel filler material. This ensures substantially complete removal of the hydrogel filler material by simply removing the balloon.

Conformable, fillable balloons in accordance with the present disclosure may be provided in a variety of shapes and dimensions depending on the target implantation site for the balloon. Balloon shapes include spheroidal balloon shapes, including spheres, various elongated balloon shapes including cylindrical balloon shapes which may have, for example, partial spheroidal ends (e.g., sausage-shaped balloons) or cone-shaped ends or prolate spheroids (e.g. football-shaped balloons), pear-shaped balloons, torus-shaped balloons (e.g., doughnut-shaped balloons), flattened balloon shapes including oblate spheroids (e.g., balloons shaped like lentils or M&M'S® candies) and other disk shaped balloons, among others.

Conformable, fillable balloons in accordance with the present disclosure include conformable, fillable balloons having a longest dimension (e.g., diameter for a sphere, the width for a disk, length for an elongated balloon such as a sausage-shaped balloon, etc.) ranging anywhere from 1 mm or less to 40 mm or more, for example, ranging from 1 mm to 2.5 mm to 5 mm to 10 mm to 20 mm to 40 mm, among other possibilities, when filled.

Conformable, fillable balloons in accordance with the present disclosure include conformable, fillable balloons having a volume ranging anywhere from 0.1 ml or less to 250 ml or more, for example, ranging anywhere from 0.1 ml to 0.2 ml to 0.5 ml to 1.0 ml to 2.5 ml to 5 ml to 10 ml to 25 ml to 50 ml to 100 ml to 250 ml, among other possibilities, when filled.

Conformable, fillable balloons in accordance with the present disclosure include conformable, fillable balloons having a wall thickness ranging anywhere from 10 or less to micrometers to 5000 micrometers or more, for example, ranging from 10 micrometers to 25 micrometers to 50 micrometers to 100 micrometers to 250 micrometers to 500 micrometers to 1000 micrometers to 5000, among other possibilities, when filled.

Conformable, fillable balloons in accordance with the present disclosure may have a feature that allows filler material to be injected into the balloon, while preventing outflow of the filler material after the balloon is filled. For example, the balloons may be provided with a check valve such as a duckbill valve that allows flow of the filler material into the balloon while preventing reverse flow of the filler material out of the balloon. As another example, the balloon may be provided with an aperture which allows flow of the filler material into the balloon and a plug that is configured to seal the aperture to prevent flow of the filler material out of the balloon once filled. As another example, the balloon may be provided with an aperture through which a small tube such as a hypotube is inserted for filling the balloon, which aperture is resealed upon withdrawal of the tube from the aperture (e.g., due to elastic recovery of the aperture or a constricting device that constricts the aperture, such as a clamp or elastic ring), preventing flow out of the balloon.

Conformable, fillable balloons in accordance with the present disclosure are formed from a variety of materials, depending on the target implantation site of the balloon, including biostable balloon materials, which remain intact and retain the hydrogel filling material for at least 20 years after implantation, and degradable materials which are configured to break down and be removed from the body after a time period ranging from 1 day to 24 months (e.g., ranging anywhere from 1 day to 3 days to 1 week to 2 weeks to 1 month to 3 months to 6 months to 12 months to 24 months). Degradation may occur due to various mechanisms that include hydrolysis by interaction with water in the body, enzymatic degradation by enzymes that are naturally occurring in the body or enzymes that are introduced into the body, oxidation by oxidants produced by the body, and/or by breakdown using cleavage compositions as described in more detail below.

Balloon materials for use herein include biostable polymeric materials and degradable polymeric materials, including biodegradable polymeric materials. Degradable polymeric materials may undergo bond cleavage, resulting in reduced molecular weight and solubilization of the smaller polymeric chains in biological fluids. The dissolved polymer chains may be completely metabolized, excreted by the kidneys, and/or absorbed via other physiological mechanisms.

Balloon materials for use herein are generally biocompatible with the space in which they are implanted. In some embodiments, the balloon materials are compliant (stretchable) polymers such as formed from elastomeric polymers. In some embodiments, the balloon materials are formed from thermoplastic polymers. In some embodiments, the balloon materials are in the form of crosslinked networks, including hydrogel crosslinked networks.

Biostable polymeric materials may be selected, for example, from the following polymers, among others: polyamides, polyolefins including polypropylene, polybutadienes including hydrogenated polybutadienes, polydimethylsiloxane (PDMS), polyurethanes, styrene block copolymers, including styrene-isobutylene-styrene triblock copolymers, styrene-isoprene-styrene triblock copolymers, styrene-butadiene-styrene triblock copolymers, and styrene-isoprene/butadiene-styrene triblock copolymers (e.g., Kraton™ polymers from Kraton Corporation, The Woodlands, TX, USA).

Degradable polymeric materials may be selected, for example, from the following polymers, among others: polyesters including polylactones such as polyvalerolactone and poly(l-lactide-co-ε-caprolactone), polycarbonates such as poly(hexamethylene carbonate) (polyHMC), polydioxane, poly(lactide co-glycolide), polyethylene glycol, polyvinyl alcohol, water soluble polyacrylates (e.g. polyacrylic acid), polyoxazolines, polypeptides and proteins.

Degradable polymeric materials may be selected, for example, from hydrophilic polymer hydrogels that comprise crosslinks between hydrophilic polymer chains within the hydrophilic polymer hydrogel. The hydrophilic polymer chains for use herein are soluble in water and can be cleared from the body, for example, by metabolic processes, clearance through the kidneys and/or other physiological processes. Various polymers for use as hydrophilic polymer chains are listed below.

As used herein, a “hydrogel” is a crosslinked polymer that contains water or can absorb water but does not dissolve when placed in water, although hydrogels may degrade in vivo over time or be degraded by introduction of ex vivo substances. Hydrogels can be crosslinked by various mechanisms include physical crosslinking and covalent crosslinking.

In some embodiments, degradable polymeric materials for use herein as balloon materials are formed from hydrophilic polymer chains that are linked to one another by crosslinks that contain hydrolysable linkers that degrade upon contact with water, including tunable-rate hydrolysable linkers such as those described below in conjunction with hydrogel filler materials for use in the present disclosure.

In some embodiments, degradable polymeric materials for use herein as balloon materials are formed from hydrophilic polymer chains that are linked to one another by crosslinks that contain reversible covalent linkages that incorporate reactive dimeric linkers, including those described below in conjunction with hydrogel filler materials for use in the present disclosure, whose degradation in situ (i.e., within a subject) is triggered by contact with a cleavage composition.

In some embodiments, degradable polymeric materials for use herein as balloon materials are formed from hydrophilic polymer chains that are linked to one another by crosslinks that contain immolative linkers, including those described below in conjunction with hydrogel filler materials for use in the present disclosure, whose degradation in situ (i.e., within a subject) is triggered by contact with a cleavage composition.

In some embodiments, the conformable, fillable balloons may be surface functionalized. For example, the conformable, fillable balloons may be surface functionalized to promote thrombus or to promote binding to tissue surfaces. For example, in some embodiments, the balloon surface may be functionalized with N-Hydroxysuccinimide (NHS) esters to bind to biomolecules including proteins in bodily fluid and surface proteins, thereby promoting thrombus formation and tissue adhesion, reducing the risk of migration of the balloon. In some embodiments, the balloon surface may be functionalized with plastic antibodies to selectively bind to proteins that promote thrombogenicity.

In other embodiments, the conformable, fillable balloons may be surface functionalized to limit thrombus. For example, in some embodiments, the balloon surface may be treated with a fluoropolymer such as polyvinylidene fluoride (PVDF) or rivaroxaban to limit thrombus formation.

Hydrogel filler materials for use herein include biostable hydrogel filler materials, which remain intact and retain the hydrogel filling material for at least 20 years after implantation, and degradable hydrogel filler materials which are configured to break down and be removed from the body after a time period ranging from 1 day to 24 months (e.g., ranging anywhere from 1 day to 3 days to 1 week to 2 weeks to 1 month to 3 months to 6 months to 12 months to 24 months). Degradation may occur due to various mechanisms that include hydrolysis by interaction with water in the body, enzymatic degradation by enzymes that are naturally occurring in the body or enzymes that are introduced into the body, oxidation by oxidants produced by the body, and/or by breakdown using cleavage compositions that are introduced into the body as described in more detail below.

In addition to crosslinked polymers, filler materials for use herein may further include one or more additional agents, including therapeutic agents, imaging agents, colorants, tonicity adjusting agents, and pH adjusting agents as described below.

In various embodiments, filler materials in accordance with the present disclosure have a radiopacity that is greater than 100 Hounsfield units (HU), beneficially ranging anywhere from 100 HU to 250 HU to 500 HU to 750 HU to 1000 HU or more, for example, when measured on bench-top micro-CT systems such as XtremeCT from Scanco Medical (Wangen-Brüttisellen, Switzerland) or similar.

Filler materials for use herein include pre-formed hydrogel filler materials that are formed ex vivo and subsequently delivered to a subject and hydrogel filler materials that are formed in situ within a subject.

Pre-formed filler materials in accordance with the present disclosure include injectable suspensions of hydrogel particles. Hydrogel particles may vary widely in size, for example, having an average size ranging from 50 to 950 microns. Hydrogel particles may be provided in a suitable reservoir, such as a syringe, vial or ampule. Whether supplied in a syringe, vial, ampule or other reservoir, the pre-formed filler compositions may be provided, for example, in dry form (e.g., in the form of a powder that contains hydrogel particles) or in a fluid form (e.g., in the form of a suspension that contains hydrogel particles).

Pre-formed hydrogel filler materials in accordance with the present disclosure further include injectable shear-thinning hydrogel filler materials. A material is a shear-thinning hydrogel filler material if the viscosity of the material decreases with increasing shear. Such materials can temporarily fluidize under shear stress and recover their original mechanical properties after release of the applied stress.

In various embodiments, the injectable shear-thinning hydrogel filler materials of the present disclosure are physically crosslinked hydrogel filler materials. In some of these embodiments, the injectable shear-thinning hydrogel filler materials of the present disclosure are physically crosslinked by ionic interactions. Having shear-thinning properties enables the filler materials to be injected as a low viscosity, flowing material. Once injection shear is removed, restoration of pre-shear filler material rigidity allows the filler material to remain localized in the balloon after injection. When not under shear the injectable shear-thinning hydrogel filler materials are generally in the form of soft solids.

In some embodiments, the injectable shear-thinning hydrogel filler materials are based on natural polymers such as fibrous proteins or polysaccharides such as hyaluronate, alginate, and chitosan. Such hydrogels are generally crosslinked based on physical crosslinking mechanisms such as electrostatic interactions or hydrogen bonding.

In particular embodiments, the injectable shear-thinning hydrogel filler materials of the present disclosure comprise a fibrous protein, silicate particles and water.

Fibrous proteins are made up of polypeptide chains that are elongated and fibrous in nature or have a sheet-like structure. Fibrous proteins for use in the injectable shear-thinning hydrogel filler materials of the present disclosure include animal-derived proteins and non-animal-derived proteins. Fibrous proteins for use in the injectable shear-thinning hydrogel filler materials of the present disclosure include keratin, elastin, fibroin, myosin, desmin, fibrin, actin, and collagen, including denatured and hydrolyzed forms thereof such as gelatin, polyarginine, polylysine, and spider silk proteins. Specific fibrous proteins for use herein include bovine collagen, porcine collagen, equine collagen, porcine gelatin (e.g., type-A porcine gelatin, gelatin derived from porcine skin, gelatin derived from porcine bones, and the like), bovine gelatin (e.g., type-B bovine gelatin, gelatin derived from bovine skin, gelatin derived from bovine bones, and the like), equine gelatin, avian-derived gelatin and fish-derived gelatin.

Silicate microparticles for use in the injectable shear-thinning hydrogel filler materials of the present disclosure include natural silicate microparticles and synthetic silicate microparticles. Particular examples of silicate microparticles include natural and synthetic silicate layered clays. Natural silicate layered clays include montmorillonite, saponite, hectorite, kaolinite, palygorskite and sepiolite, among others. Synthetic silicate layered clays include lithium magnesium sodium silicates such as Laponite®-based silicate nanoplatelets (e.g., Laponite® XLG-based silicate nanoplatelets, Laponite® XLS-based silicate nanoplatelets, Laponite® XL21-based silicate nanoplatelets, and Laponite® D-based silicate nanoplatelets), Sumecton® SWN and Lucentite™ SWN, magnesium aluminum silicates such as Sumecton® SA, sodium magnesium silicates such as Optigel® SH and SUPLITE-MP, and fluoromica such as Somasif™ ME100, among others.

The silicate microparticles for use in the injectable shear-thinning hydrogel filler materials of the present disclosure include microparticles that have a neutral charge, microparticles that have a net positive charge, and microparticles that have a net negative charge. The net charge of the silicate microparticles may depend upon the pH of the injectable shear-thinning hydrogel filler material. In some embodiments, the silicate microparticles have a net positive charge at the pH of the injectable shear-thinning hydrogel filler material. In some embodiments, the silicate microparticles have a negative positive charge at the pH of the of the injectable shear-thinning hydrogel filler material.

In some embodiments, the pH of the of the injectable shear-thinning hydrogel filler material pH between 6 and 11, more typically between 8 and 10.

In some embodiments, the silicate microparticles are plate-shaped. In some embodiments, the silicate microparticles are silicate layered clays characterized by a discotic charge distribution on the surface. In some embodiments, the plate-shaped silicate microparticles comprise a positively charged edge and a negatively charged surface. In some embodiments, the overall charge of the silicate microparticles is negative. In some embodiments, the plate-shaped silicate microparticles are from about 5 nm to about 60 nm in diameter, for example, from about 10 nm to about 40 nm in diameter, from about 10 nm to about 30 nm in diameter, or from about 20 to about 30 nm in diameter. In some embodiments, the plate-shaped silicate microparticles are from about 0.5 nm to about 2 nm in thickness, or about 1 nm in thickness.

In some embodiments, the injectable shear-thinning hydrogel filler materials further comprise one or more additional agents, including therapeutic agents, imaging agents, colorants, tonicity adjusting agents, and pH adjusting agents as described below.

Such additional agents include radiopaque additives such as radiopaque microparticles that contain radiopaque metals and/or radiopaque metal compounds. Such radiopaque microparticles may be spherical or non-spherical. For example, the radiopaque microparticles may contain a radiopaque metal or metal compound throughout, may contain a cladding of a radiopaque metal or metal compound that surrounds a core of another material such as stainless steel, or may contain a core of a radiopaque metal or metal compound surrounded by a cladding of another material such as stainless steel. Radiopaque metals include transition metals such as tantalum, gold, platinum, tungsten, and alloys containing one or more of the same. Radiopaque metal compounds include compounds of barium, tantalum, bismuth, or gold such as barium sulfate, tantalum oxide, bismuth oxide, bismuth subcarbonate, bismuth oxychloride, gold oxide, tungsten oxide, and tungsten carbide. Additional examples of radiopaque additives include non-ionic radiopaque additives, such as iohexol, iodixanol, ioversol, iopamidol, ioxilan, or iopromide, ionic radiopaque additives such as diatrizoate, iothalamate, metrizoate, or ioxaglate, and iodinated oils, including ethiodized poppyseed oil (available as Lipiodol®).

One currently available injectable shear-thinning hydrogel filler material is Obsidio™ Conformable Embolic from Boston Scientific Corporation, Marlborough, Massachusetts, USA. It is pre-packaged in a ready-to-use syringe. As the material is pushed through a tube such as a catheter on its way to a targeted site during administration, the material shear-thins and flows readily, like a liquid. When shear forces are removed as the material reaches its intended location, it reverts to a soft solid. The Obsidio™ embolic hydrogel filler material contains laponite, gelatin, water and tantalum. The Obsidio™ material is radiopaque due to the presence of tantalum particles in its formulation.

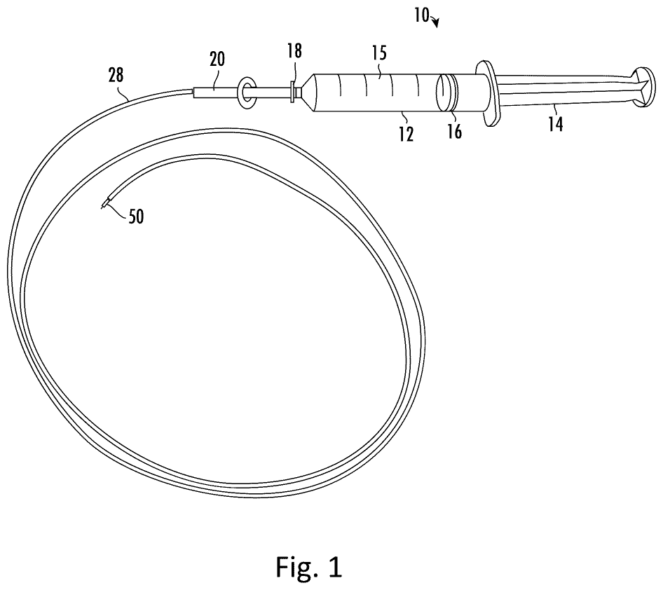

FIG. 1 illustrates an apparatus that can be employed to fill a conformable, fillable balloon in accordance with the present disclosure. The apparatus includes a syringe 10 that includes a barrel 12, a plunger 14, and one or more stoppers 16. The barrel 12 may include a suitable adapter/connector such as a Luer adapter, e.g., at the distal end 18 of the barrel 12, for attachment to a flexible catheter 29. The proximal end of the catheter 29 may include a suitable adapter/connector 20 for receiving the adapter/connector of the barrel 12. In some embodiments, the distal end of the catheter 29 may be removably attached to a conformable, fillable balloon as described herein. The syringe barrel 12 serves as a reservoir for a pre-formed hydrogel filler material to be injected through the catheter.

In some embodiments the hydrogel filler materials for use herein are formed in vivo.

In some of these embodiments, hydrogel filler materials in accordance with the present disclosure also may be in the form of a lower critical solution temperature (LCST) material, where the LCST refers to the temperature at which the transition from a liquid phase to a hydrogel phase occurs. When the LCST material is injected into a body, the temperature of the LCST material is below the LCST and increases due to heat transfer from the body. Once the temperature of the LCST material reaches the LCST, the transition from the liquid phase to the hydrogel phase takes place. Such LCST materials can be configured or prepared to remain in a liquid phase, with a low viscosity, at an injection temperature below the body temperature (e.g., room temperature or a chilled temperature below room temperature) and to transform to a gel phase when increased in temperature to body temperature. Examples of such materials include polyoxyethylene-polyoxyproplyene (PEO-PPO) block copolymers, such as Pluronic F127 and F108, and N-Isopropyl acrylamide (IPAAm) copolymers. Such hydrogel filler materials may be administered using a device like that of FIG. 1, among other possibilities

In some of these embodiments, hydrogel filler materials in accordance with the present disclosure may form covalently crosslinked hydrogels in vivo. In such embodiments, hydrogel precursors are provided that, when combined, produce a hydrogel filler material in the balloon in vivo. Hydrogel precursors may be provided in a suitable reservoir, such as a syringe, vial or ampule. Whether supplied in a syringe, vial, ampule or other reservoir, the hydrogel precursors may be provided, for example, in dry form (e.g., in the form of a powder that contains one or more hydrogel precursors) or in a fluid form (e.g., in the form of a solution that contains one or more hydrogel precursors).

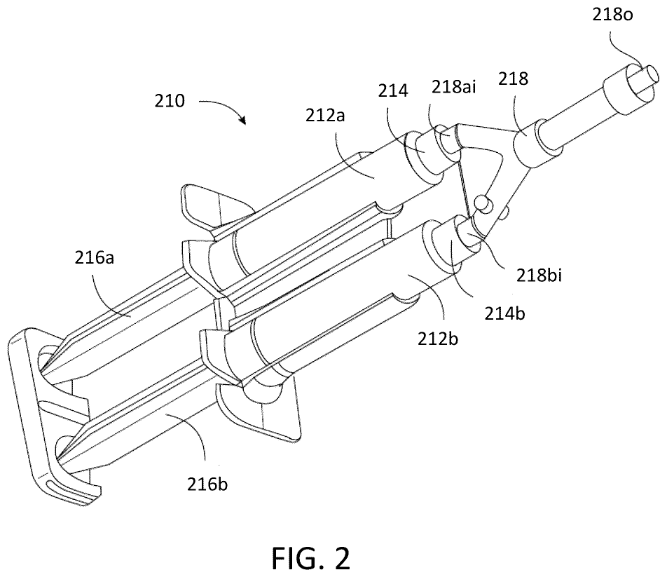

One example of such a crosslinked hydrogel is SpaceOAR®, which is based on a multi-arm polyethylene glycol (PEG) polymer functionalized with succinimidyl glutarate as activated end groups as a first precursor which further react with trilysine as a second precursor to form covalent crosslinks. During use, a solution of the multi-arm polymer and the lysine is simultaneously injected with a buffer solution. When mixed, the buffer solution increases the pH and dramatically accelerates the rate of reaction between the multi-arm polymer and the lysine, forming a crosslinked hydrogel within seconds. The buffer solution and the solution of the multi-arm polymer and the lysine can be simultaneously injected using a device like that described in FIG. 2 below. Another example of such a crosslinked hydrogel is SpaceOAR Vue®, which, like SpaceOAR®, is based on a multi-arm polyethylene glycol (PEG) polymer functionalized with succinimidyl glutarate as activated end groups which further react with trilysine to form crosslinks. In SpaceOAR Vue®, however, some of the succinimidyl glutarate end groups are functionalized with 2,3,5-triiiodobenzamide groups, providing radiopacity. Further examples of such multi-precursor systems are described below.

In some embodiments, multi-precursor systems can be used to form hydrogels ex vivo, after which the hydrogels are broken down into particles and suspended in an aqueous solution to form an injectable pre-formed hydrogel.

In some embodiments, the filler materials of the present disclosure can be imaged after administration using a suitable imaging technique such as ultrasound or an X-ray-based imaging technique, such as computerized tomography or X-ray fluoroscopy.

Filler materials in accordance with the present disclosure further include those that are formed from hydrophilic polymer hydrogels that comprise covalent crosslinks between hydrophilic polymer chains within the hydrophilic polymer hydrogels, which crosslinks contain hydrolysable linkers that are dispersed throughout the hydrophilic polymer hydrogels. Such hydrophilic polymer hydrogels can be degraded in situ by hydrolysis, among other possible mechanisms.

In some embodiments, the hydrolysable linkers may be selected from the following groups, among others: a carboxylic acid ester linkage,

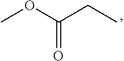

a carbonate linkage,

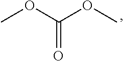

an acid anhydride linkage,

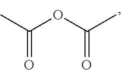

an imide linkage,

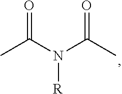

a ketal linkage,

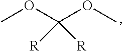

a carbamate linkage,

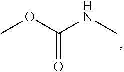

an organophosphate ester linkage,

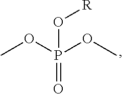

a silane linkage,

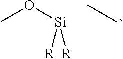

an amide linkage,

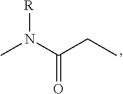

a hydrozonium linkage,

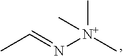

an acylhydrozone linkage,

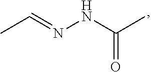

an oxime linkage,

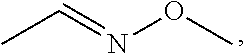

or an amidohydrozone linkage,

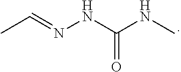

In some embodiments, the crosslinks may further comprise an activating group that is positioned proximate to the hydrolysable linker in order to tune the rate of hydrolysis of the hydrolysable linker and, in particular embodiments, increase the rate of hydrolysis of the hydrolysable linker. Examples of activating groups include electron donating activating groups and electron withdrawing activating groups that can promote hydrolysis, either through binding and orienting water for subsequent attack, or through binding directly to the hydrolysable linker to accelerate and tune the reaction rate. These activating groups can function, for example, via hydrogen bond donation, electrostatic stabilization, metal/ligand interactions, and/or lone-pair donation, among other mechanisms.

Particular examples of activating groups include hydrogen bond donor/acceptors or lone pair donors, which can complex with hydrogen bond acceptors/donors or lone pair acceptors. In this regard, it is noted that water is both a hydrogen bond donor and a hydrogen bond acceptor, with each water molecule having two lone pairs that can serve as hydrogen bond acceptors and two O—H bonds that provide a pair of hydrogen bond donors. Particular examples of such activating groups are ureas, thioureas, alcohols and amines, which may be provided in the form of a urea linkage,

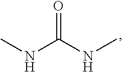

a urea pendant group,

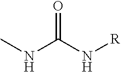

where R is H, a thiourea linkage,

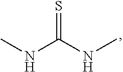

a thiourea pendant group,

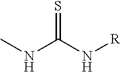

where R is H, an amine linkage,

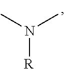

where R is H, an amine pendant group,

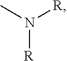

where R is H, and an alcohol pendant group, such as a hydroxyalkyl group having from 1 to 7 carbon atoms.

Particular examples of activating groups also include lone pair acceptors in the form of uncharged moieties that can complex with lone pair donors, including water molecules. Particular examples of such activating groups are silicon- and boron-containing groups, for example, and may be provided in the form of a silicon-containing linkage,

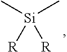

where R is methyl, a silicon-containing pendant group,

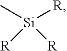

where R is methyl, a boron-containing linkage,

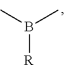

where R is an aryl or alkyl group, and a boron-containing pendant group

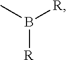

where R is an aryl or alkyl functional group, most favorably an electron deficient aryl or alkyl group.

Particular examples of activating groups further include water complexing or electrostatic stabilizers (e.g., charged moieties, which can position water in an adjacent position). Particular examples of such activating groups are phosphonate and sulfonate groups, which may be provided in the form of a phosphonate linkage,

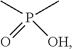

a phosphonate pendant group,

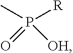

where R is OH, alkyl or aryl, and a sulfonate pendant group,

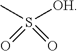

In some embodiments, the activating groups are incorporated such that they complex with the hydrolysable linker in a cyclic transition state, specifically, a 4-9 atom cyclic transition state. For example, the activating groups may be appended via aromatic ring spacers, cycloaliphatic ring spacers, aliphatic spacers, or heteroatom spacers. This spacing, leading to a 4-9 atom cyclic transition state, may be 1-6 atoms in length.

Polymer chains for use herein may be selected from any of a variety of synthetic, natural, or hybrid synthetic-natural polymer chains. Examples of polymer chains include those that are formed from one or more monomers selected from the following, among others: C₁-C₆-alkylene oxide monomers (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), and 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer chains may be selected, for example, from the following polymer chains, among others: polyether chains including poly(C₁-C₆-alkylene oxide) chains such as poly(ethylene oxide) (PEO) chains (also referred to as polyethylene glycol chains or PEG chains), poly(propylene oxide) chains, poly(ethylene oxide-co-propylene oxide) chains, poly(tetramethylene oxide) chains, polyester chains including polyglycolide chains, polylactide chains, poly(lactide-co-glycolide) chains, poly(β-propiolactone) chains, poly(β-butyrolactone) chains, poly(γ-butyrolactone) chains, poly(γ-valerolactone) chains, poly(δ-valerolactone) chains, and poly(ε-caprolactone) chains, polyoxazoline chains including poly(2-C₁-C₆-alkyl-2-oxazoline chains) such as poly(2-methyl-2-oxazoline) chains, poly(2-ethyl-2-oxazoline) chains, poly(2-propyl-2-oxazoline) chains, poly(2-isopropyl-2-oxazoline) chains, and poly(2-n-butyl-2-oxazoline) chains, poly(2-phenyl-2-oxazoline) chains, polymer chains formed from one or more polar aprotic vinyl monomers, including poly(N-vinyl pyrrolidone) chains, poly(acrylamide) chains, poly(N-methyl acrylamide) chains, poly(dimethyl acrylamide) chains, poly(N-vinylimidazole) chains, poly(4-vinylimidazole) chains, and poly(sodium 4-vinylbenzenesulfonate) chains, polydioxanone chains, poly(N-isopropylacrylamide) chains, and polypeptide chains.

Polymer chains for use in the present disclosure typically contain between 10 and 20000 monomer units or more, for example, ranging from 10 monomer units to 20 monomer units to 50 monomer units to 100 monomer units to 200 monomer units to 500 monomer units to 1000 monomer units to 5000 monomer units to 10000 monomer units to 20000 monomer units, among other possibilities.

In some embodiments, systems for forming hydrogel filler materials are provided that comprise (a) a reactive multi-arm polymer comprising a core region and a plurality of polymer arms having reactive moieties, each polymer arm comprising a polymer chain linked to the core region and one of the plurality of reactive moieties linked to the polymer chain through a hydrolysable linkage, which may be selected from a carbonate linkage, an acid anhydride linkage, an imide linkage, a ketal linkage, a carbamate linkage, an organophosphate ester linkage, a silane linkage, an amide linkage, a hydrozonium linkage, an acylhydrozone linkage, an oxime linkage and an amidohydrozone linkage as described above and (b) a multifunctional crosslinking compound comprising a plurality of complementary reactive moieties that are reactive with the reactive moieties of the reactive multi-arm polymer. In some of these embodiments, an activating group such as is described above, is positioned adjacent to the hydrolysable linkage, which increases a rate of hydrolysis of the hydrolysable linkage.

In some embodiments, systems for forming hydrogel filler materials are provided that comprise (a) a reactive multi-arm polymer comprising a core region and a plurality of polymer arms having reactive moieties, each polymer arm comprising a polymer chain linked to the core region and one of the plurality of reactive moieties linked to the polymer chain and (b) a multifunctional crosslinking compound comprising a plurality of complementary reactive moieties that are reactive with the reactive moieties of the reactive multi-arm polymer, wherein the plurality of complementary reactive moieties are each linked to a remainder of the multifunctional crosslinking compound through a hydrolysable linkage, which may be selected from a carbonate linkage, an acid anhydride linkage, an imide linkage, a ketal linkage, a carbamate linkage, an organophosphate ester linkage, a silane linkage, an amide linkage, a hydrozonium linkage, an acylhydrozone linkage, an oxime linkage and an amidohydrozone linkage as described above. In some of these embodiments, the multifunctional crosslinking compound further comprises an activating group positioned adjacent to each hydrolysable linkage, which increases a rate of hydrolysis of the hydrolysable linkage.

In some embodiments, the reactive moieties and complementary reactive moieties may be selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups, among others.

For example, a crosslinked reaction product can be formed from the following: a reactive multi-arm polymer having electrophilic groups and a multifunctional crosslinking compound having nucleophilic groups; a reactive multi-arm polymer having nucleophilic groups and a multifunctional crosslinking compound having electrophilic groups; a reactive multi-arm polymer having diene groups and a multifunctional crosslinking compound having dienophilic groups; a reactive multi-arm polymer having dienophilic groups and a multifunctional crosslinking compound having diene groups; a reactive multi-arm polymer having strained alkyne groups and a multifunctional crosslinking compound having azide groups; a reactive multi-arm polymer having azide groups and a multifunctional crosslinking compound having strained alkyne groups; a reactive multi-arm polymer having strained alkene groups and a multifunctional crosslinking compound having tetrazine groups; a reactive multi-arm polymer having tetrazine groups and a multifunctional crosslinking compound having strained alkene groups; a reactive multi-arm polymer having alkene groups and a multifunctional crosslinking compound having thiol groups; and a reactive multi-arm polymer having thiol groups and a multifunctional crosslinking compound having alkene groups.

Electrophilic groups may be selected, for example, from cyclic imide ester groups, such as succinimide ester groups,

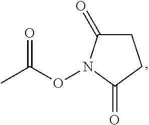

maleimide ester groups, glutarimide ester groups, diglycolimide ester groups, phthalimide ester groups, and bicyclo[2.2.1]hept-5-ene-2,3-dicarboxylic acid imide ester groups,

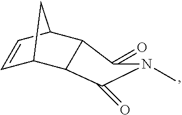

imidazole ester groups, imidazole carboxylate groups and benzotriazole ester groups, among other possibilities. Nucleophilic groups may be selected, for example, from amine groups, thiol groups, and hydroxyl groups, among other possibilities. Strained alkyne groups may be selected, for example, from (1R,8S,9s)-bicyclo[6.1.0]non-4-yn-9-yl groups,

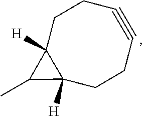

and dibenzocyclooctyne groups, among other possibilities. Strained alkene groups may be selected, for example, from cyclooct-4-en-1-yl groups,

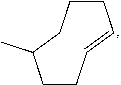

cyclooct-4-enyl 2,5-dioxopyrrolidin-1-yl carbonate groups, carbamic acid, N-(3-aminopropyl)-, 4-cycloocten-1-yl ester groups, or carbamic acid, N-[2-[2-(2-aminoethoxy) ethoxy]ethyl]-, 4-cycloocten-1-yl ester groups, among other possibilities.

General classes of core regions include residues of polyols, including sugars (monosaccharides, disaccharides, trisaccharides, etc.) and sugar alcohols, calixaranes, polyhedral oligomeric silsesquioxanes (POSS), cyclodextrin, polyhydroxylated polymers, catechins, flavanols, anthocyanins, stilbenes, and polyphenols, among many others.

As previously noted, hydrogel filler materials for use herein include those that are formed in situ within a subject. FIG. 2 illustrates a system that can be employed to fill a conformable, fillable balloon with hydrogel filler material that is formed in situ, in accordance with an embodiment of the present disclosure. The system may include a delivery device 210 that comprises a double-barrel syringe, which includes a first barrel 212a having a first barrel outlet 214a, which first barrel contains a first composition comprising a reactive multi-arm polymer as described herein, a first plunger 216a that is movable in the first barrel 212a, a second barrel 212b having a second barrel outlet 214b, which second barrel 212b contains a second composition that contains a multifunctional crosslinking compound as described herein, and a second plunger 216b that is movable in the second barrel 212b. In some embodiments, the device 210 may further comprise a mixing section 218 having a first mixing section inlet 218ai in fluid communication with the first barrel outlet 214a, a second mixing section inlet 218bi in fluid communication with the second barrel outlet, and a mixing section outlet 2180. The mixing section outlet 2180 may include a suitable adapter/connector such as a Luer adapter for attachment to a flexible catheter like that shown in FIG. 1. In some embodiments a distal end of the flexible catheter is attached to a conformable, fillable balloon as described herein.

During operation, when the first and second plungers are depressed, the first and second fluid compositions are dispensed from the first and second barrels, whereupon the first and second fluid compositions mix, after which the reactive multi-arm polymer and the multifunctional crosslinking compound ultimately crosslink to form a crosslinked hydrogel filler material in the balloon. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. A catheter tube is attached to the mixing section outlet, allowing a conformable, fillable balloon to be filled with the admixture after passing through the catheter tube.

Filler materials in accordance with the present disclosure further include triggerable-degradation filler materials that are formed from hydrophilic polymer hydrogels that comprise crosslinks between polymer chains within the hydrophilic polymer hydrogels, which crosslinks contain reversible covalent linkages that incorporate reactive dimeric linkers or contain immolative linkers that are dispersed throughout the hydrophilic polymer hydrogels. Such hydrophilic polymer hydrogels can be degraded in situ by contacting the hydrophilic polymer hydrogels with a cleavage composition that contains a singly reactive molecule, which acts to break the crosslinks at the positions of the dimeric reactive linkers or the immolative linkers within the crosslinks.

As used herein an “immolative linker” is defined as a transient covalent bond that can be cleaved through the addition of a triggering molecule.

As used herein a “singly reactive compound” is defined as a compound that will only undergo one reaction with a desired substrate.

In some embodiments, the hydrophilic polymer hydrogel comprises crosslinks between hydrophilic polymer chains within the hydrophilic polymer hydrogel, which crosslinks contain reversible covalent linkages that incorporate reactive dimeric linkers, and the hydrophilic polymer hydrogel may be degraded by contacting the hydrophilic polymer hydrogel with a cleavage composition that contains a singly reactive molecule, which acts to break the crosslinks at the position of the dimeric linkers within the crosslinks.

The cleavage compositions of the present disclosure may be supplied in a syringe, vial, ampule or other reservoir. The cleavage compositions may be provided, for example, in dry form (e.g., powder form) or in fluid form, such as in a solution form or as an aqueous dispersion.

The cleavage compositions may further include one or more additional agents, including therapeutic agents, imaging agents, colorants, tonicity adjusting agents, and pH adjusting agents as described below.

Contact of the cleavage composition with the hydrophilic polymer hydrogel may include, for example, applying the cleavage composition onto a surface of the hydrogel, injecting the cleavage composition into the hydrogel, and so forth.

With regard to hydrogels having crosslinks that contain reversible covalent linkages that are based on reactive dimeric linkers, a general schematic representation is provided below, which shows two reversible covalent linkages, represented by X-Y. The covalent linkages. X-Y, reversibly break down resulting in (a) a dimeric reactive linker, represented by X-B-X, where B represents a polymer chain and where X represents a reactive group attached to the polymer chain, B, and (b) two polymer chains, represented by AY, where Y represents a reactive group attached to a polymer chain, represented by A, that forms reversible covalent bonds with the reactive X groups of the dimeric reactive linker X-B-X. When a singly reactive molecule, represented by X-D, is introduced, the singly reactive molecule can react with Y groups of each of the AY polymer chains to form further polymer chains, represented by A-Y-X-D, which do not reversibly react with the dimeric reactive linker, X-B-X. In doing so, the singly reactive molecule, X-D, outcompetes the reverse reaction of the dimeric reactive linker, X-B-X, for the polymer chains, AY, which would otherwise reform the reversible covalent linkages. The overall result of this process is that the singly reactive molecule, XD, acts to cleave the reversible covalent linkages.

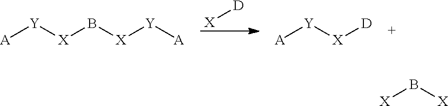

Several specific examples follow. In a first specific example, a crosslink that contains two thioester linkages as reversible covalent linkages is shown being cleaved with a singly reactive molecule, cysteine, or another thiol containing small molecule, to yield two polymer chains, each having a thioester group, and a telechelic dithiol reactive linker having two-SH groups:

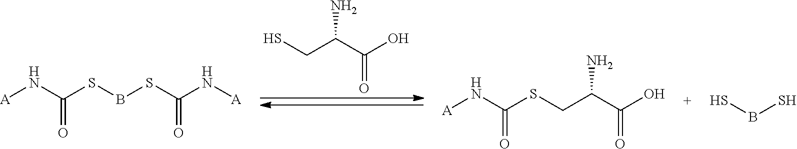

In another specific example, a crosslink that contains two disulfide linkages as reversible covalent linkages is shown being cleaved by a singly reactive molecule, such as cysteine, or another thiol containing small molecule, to yield two polymer chains, each with a disulfide group and a dithiol dimeric reactive linker having two-SH groups:

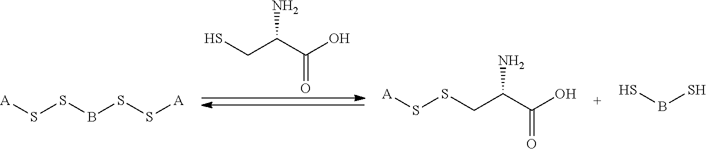

In another specific example, a crosslink that contains two linkages composed of disuccinimide as reversible covalent linkages is shown which reversibly break down into a bismaleimide type reactive linker and two polymer chains, each containing a furyl group. The polymer chains containing the furyl group then react with a singly reactive molecule, poly(N-malimide) to form two polymer chains, each with a succinimide group:

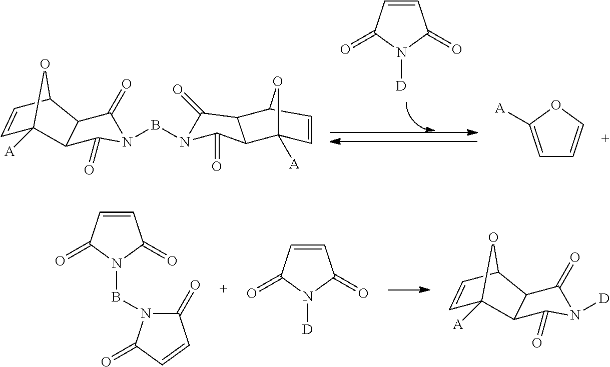

In another specific example, a crosslink that contains two borate ester linkages as reversible covalent linkages is shown which reversibly breaks down into a dimeric reactive linker containing two vicinal diol groups, and two polymer chains, each containing a boronic acid group. The polymer chains then react with a singly reactive molecule, containing a vicinal diol, to form two polymer chains, each with borate ester groups:

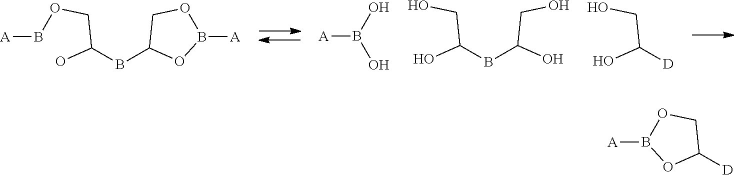

In another specific example, a crosslink that contains two linkages as reversible covalent linkages is shown which reversibly breaks down into a dimeric reactive linker containing two primary amine groups and two polymer chains, each containing an aldehyde group. The polymer chains then react with a singly reactive molecule, for example, lysine, or another primary amine containing small molecules, to form two polymer chains:

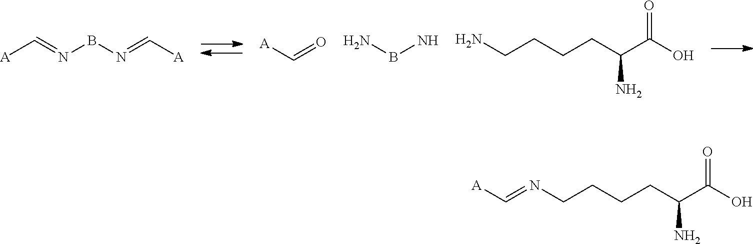

Turning now to hydrophilic polymer hydrogel having crosslinks that contain immolative linkers, several schematic representations of crosslinks between polymer chains follow, which show an immolative linker disposed between two polymer chains, each represented by the letter A, within a crosslinked hydrophilic polymer hydrogel. As will be appreciated from the description to follow, in various embodiments, instead of a single immolative linker as schematically represented, two immolative linkers may be disposed between the chains, with one immolative linker attached to each polymer chain, which immolative linkers are linked to one another through a crosslinker residue. The schematic representations also illustrate the introduction of a singly reactive molecule, which acts to cleave the immolative linker and therefore cleaves the polymer chains from one another.

In one example, a crosslink that contains an imidosydnone group as an immolative linker is shown being cleaved by a strained alkyne reactive molecule to yield one polymer chain with a primary amide group and another polymer chain with a tricyclic group:

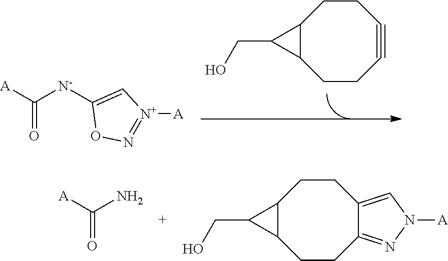

In another example, a crosslink that contains a 1-(methyloxidoamino) cyclooctene group as an immolative linker is shown being cleaved by a diboron reactive molecule to yield one polymer chain with a hydroxyl group and another polymer chain with an imminium group:

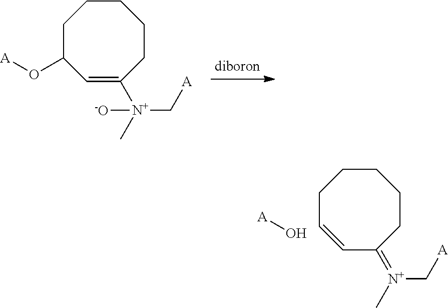

In another example, a crosslink that contains a silyl diether group as an immolative linker, where R is an alkane, cycloalkane, or aromatic side chain is shown being cleaved by a singly reactive molecule, aminophenylethyl trifluoroborate to yield two polymer chains with hydroxyl groups:

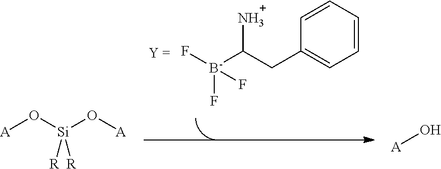

In another example, a crosslink that contains an immolative linker is shown being cleaved by a singly reactive molecule, benzene-1,2-dithiol, to yield one polymer chain with an amine or alcohol group and another polymer chain with a dithiobenzene incorporated into the final aromatic end group of the polymer chain:

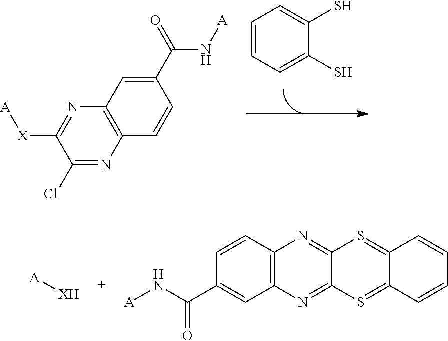

In another example, a crosslink that contains a tetrazine group as an immolative linker is shown being cleaved by a singly reactive molecule, such as a derivatized transcyclooctene, where R is an alkyl group or a 3-hydroxy-cyclooct-1-yne, to yield one polymer chain with a primary amine group and another polymer chain with a pyridazine group:

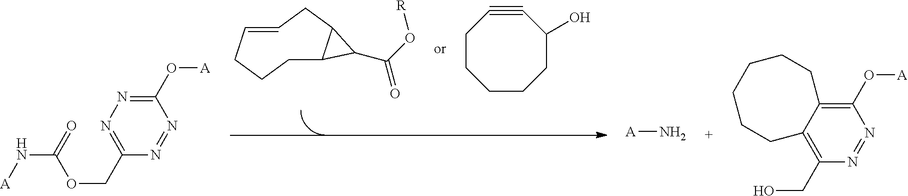

In another example, a crosslink that contains a 4-azidobenzene group as an immolative linker is shown being cleaved by a singly reactive molecule, such as (E)-cyclooct-4-enol, or through reaction with phenyl-2-carboxyl diphenylphosphine to yield one polymer chain with a carboxylate group and another polymer chain with an oxidized aromatic group bound to an amide:

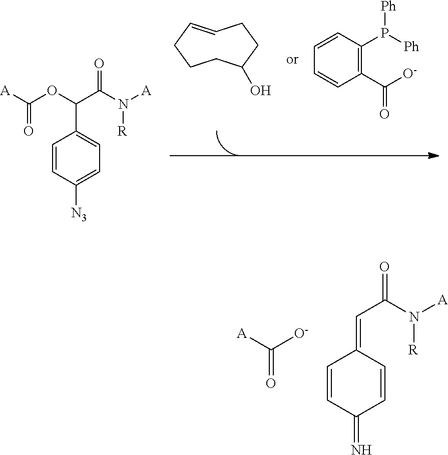

In another example, a crosslink that contains a transcyclooctene group as an immolative linker is shown being cleaved by a singly reactive molecule, such as a tetrazine derivative, where R1 and R2 are alkyl functional groups, to yield one polymer chain with a primary amine group and another polymer chain with a pyridazine group:

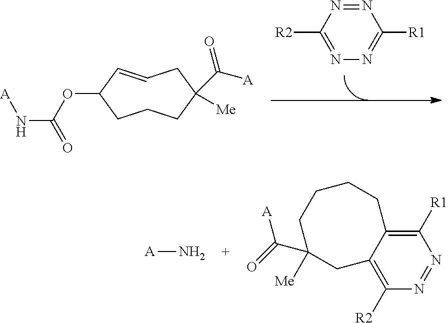

In another example, a crosslink that contains an aryl enol ether group as an immolative linker is shown being cleaved by a singly reactive molecule, such as a tetrazine derivative, where R1 and R2 are alkyl functional groups, to yield one polymer chain with a hydroxyl group and another polymer chain with an alpha beta unsaturated cyclic ketone:

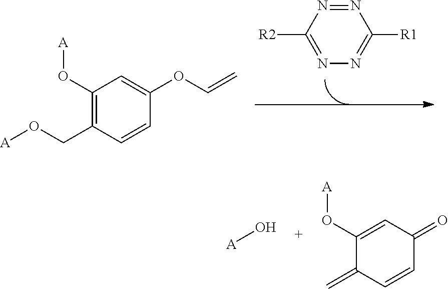

In another example, a crosslink that contains an 1,1 azide ether group as an immolative linker is shown being cleaved by a singly reactive phosphine derivative where R is alkyl or aryl, to yield one polymer chain with an aldehyde group and another polymer chain with a hydroxyl group:

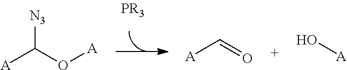

Polymer chains for use herein, including those schematically represented in the preceding schemes with the letter “A,” may be selected from any of a variety of synthetic, natural, or hybrid synthetic-natural polymer chains. Particular examples of polymer chains include those described above, among others.

In various embodiments, polymer chains, including those schematically represented in the preceding schemes with the letter A, are part of a multi-arm polymer residue where three or more polymer arms that comprise the polymer chains extend from a core region. The multi-arm polymers have three or more polymer arms (e.g., between three and fifteen polymer arms). General classes of core regions include residues of polyols, including sugars (monosaccharides, disaccharides, trisaccharides, etc.) and sugar alcohols, calixaranes, polyhedral oligomeric silsesquioxanes (POSS), cyclodextrin, polyhydroxylated polymers, catechins, flavanols, anthocyanins, stilbenes, and polyphenols, among many others.

In various embodiments, the polymer chains are crosslinked by residues of multifunctional crosslinkers that contain two or more functional groups (e.g., between two and ten functional groups).

In some of these embodiments, the multifunctional crosslinkers are dimeric reactive linkers, and functional groups at the ends of the polymer chains are reversibly crosslinked with the dimeric reactive linkers as described above.

In some of these embodiments, the multifunctional crosslinkers have functional groups that irreversibly crosslink with functional groups at ends of the polymer chains. For example, the multifunctional crosslinkers may have functional groups that irreversibly crosslink with functional groups at ends of the polymer chains, forming crosslinks that contain immolative linkers as described above. Two examples will be described in more detail here.

The first example provides hydrophilic polymer hydrogels that have crosslinks that contain imidosydnone groups as immolative linkers. In the presence of strained alkynes, imidosyndones will rapidly undergo sydnone-alkyne click chemistry reactions.

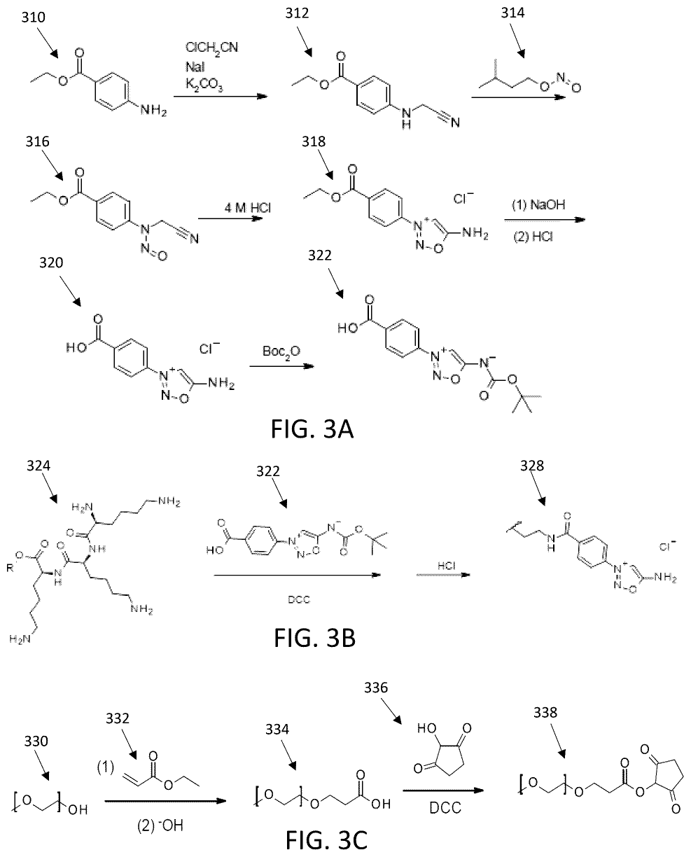

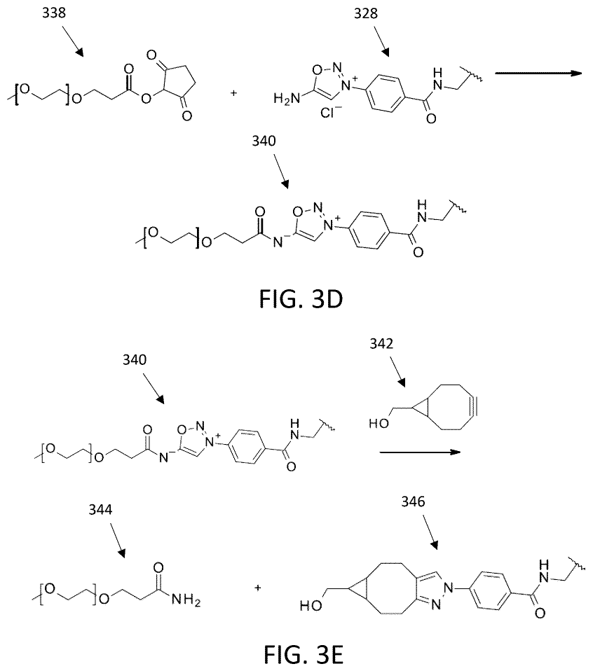

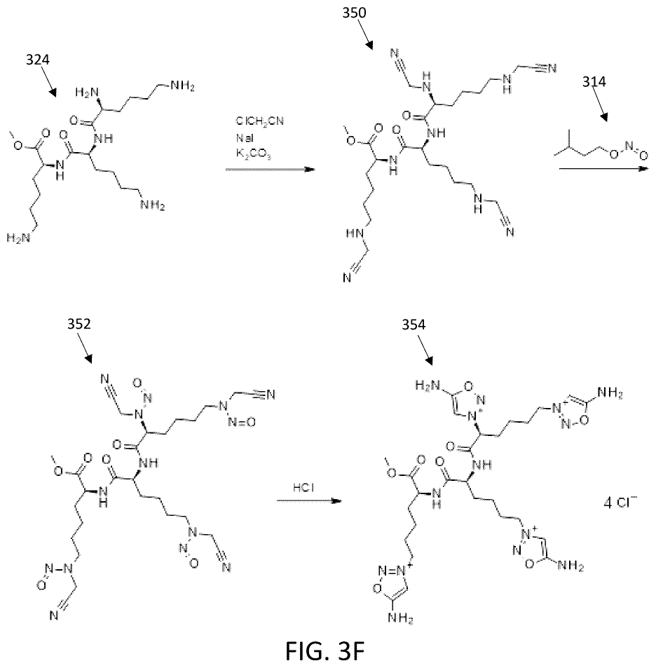

With reference now to FIG. 3A, in a first step, the amino group of 4-aminobenzoic acid methyl ester (310) is cyanomethylated with chloroacetonitrile in the presence of NaI and K₂CO₃ to yield 4-cyanomethylaminobenzoic acid methyl ester (312), which is then reacted with iso-amyl nitrite (314) to yield intermediate compound (316), followed by treatment with 4N HCl to form 4-(2-aminonitrosoethylnitrile) benzoic acid methyl ester (318), which is then sequentially treated with NaOH and HCl to form 4-imidosyndonebenzoic acid chloride (320), which is further reacted with di-tert-butyl dicarbonate (Boc₂O) to form boc-protected 4-imidosyndonebenzoic acid chloride (322).

Turning to FIG. 3B, a trilysine alkyl ester (324), where R is an alkyl group, heterocycle group, etc., that does not contain a carboxylic acid, halide, amine, alcohol, or other functional groups that can interfere with the following coupling reaction, is reacted with boc-protected 4-imidosyndonebenzoic acid methyl ester chloride (322) from FIG. 3A in the presence of a carbodiimide coupling agent such as N,N′-dicyclohexylcarbodiimide (DCC), followed by treatment in HCl to form imidosydnone-functionalized trilysine (328). Note that the terminus of only a single functionalized side chain of the three side chains of the trilysine is shown. Although trilysine is used as a multi-functional amine in this specific instance, other multi-functional amines may be used including 1,3-propanediamine, tris(3-aminopropyl)amine, 3-(2-aminoethyl) pentane-1,5-diamine, N,N′,N′-tetrakis(2-aminoethyl)-1,2-ethanediamine, 1,3,5-tris-(2-aminoethyl)-[1,3,5]triazinane-2,4,6-trione, N,N,N′-Tris(2-aminoethyl)ethylenediamine, and adamantane-1,3,5,7-tetraamine, among many others.

With reference to FIG. 3C, a hydroxyl-terminated multi-arm PEG (330) (a terminus of only one arm is illustrated) is reacted with acrylic acid ethyl ester (332) followed by treatment with base to yield a carboxyl-terminated multi-arm PEG (334), which is reacted with N-hydroxy succinimide (336) in the presence of a carbodiimide coupling agent such as DCC to yield succinimidyl-ester-terminated multi-arm PEG (338). Although a hydroxyl-terminated multi-arm PEG is employed in FIG. 3C, it will be appreciated that other hydroxyl terminated multi-arm polymers may be used including those containing the polymer chains described above.

With reference to FIG. 3D, the activated succinimidyl ester groups of the succinimidyl-ester-terminated multi-arm PEG (338) of FIG. 3C can be rapidly reacted with the primary amine groups of the imidosydnone-functionalized trilysine (328) of FIG. 3B to form a crosslinked hydrophilic polymer hydrogel (340) with crosslinks that contain imidosyndoneamide linkages.

Because the resulting hydrophilic polymer hydrogel contains crosslinks that comprise sydnone groups, which connect the multi-arm polymer residue and the trilysine-based crosslinker residue, when the hydrophilic polymer hydrogel is no longer desired, it can be degraded on demand by treatment with a strained alkyne. In a particular example shown in FIG. 3E, the crosslinked hydrophilic polymer hydrogel (340) of FIG. 3D is contacted with a strained alkyne, in particular, (1R,8S,9s)-bicyclo[6.1.0]non-4-yn-9-ylmethanol, also known as BCN—OH, thereby breaking the crosslinks, and forming a free amido-terminated arm (344), which is associated with a residue of the incorporated multi-arm PEG, and a cyclooctapyrazole group (346), which is associated with a residue of the incorporated trilysine-based crosslinker. Although BCN—OH is used in this particular example, strained alkynes can be functionalized with different groups or added to polymers to increase their biocompatibility.

As an alternative to the synthesis shown in FIGS. 3A and 3B, and with reference to FIG. 3F, in a first step the amino groups of the methyl ester of trilysine (324), or amino groups of any other suitable polyamine compound, is cyanomethylated with chloroacetonitrile in the presence of NaI and K₂CO₃ to yield cyanomethylamino functionalized trilysine (350) in which the groups of the trilysine have been converted to cyanomethylamino groups. The cyanomethylamino groups of the cyanomethylamino functionalized lysine (350) are then reacted with iso-amyl nitrite (314) to a further yield intermediate compound (352), followed by treatment with 4N HCl to form imidosydnone-functionalized trilysine (328). Although trilysine is used as a multi-functional amine in this specific instance, other multi-functional amines may be used including 1,3-propanediamine, tris(3-aminopropyl)amine, 3-(2-aminoethyl) pentane-1,5-diamine, N,N′,N′-tetrakis(2-aminoethyl)-1,2-ethanediamine, 1,3,5-tris-(2-aminoethyl)-[1,3,5]triazinane-2,4,6-trione, N,N,N′-Tris(2-aminoethyl)ethylenediamine, and adamantane-1,3,5,7-tetraamine, among many others.

The second example is based on bioorthogonal click and release chemistry using a diborane.

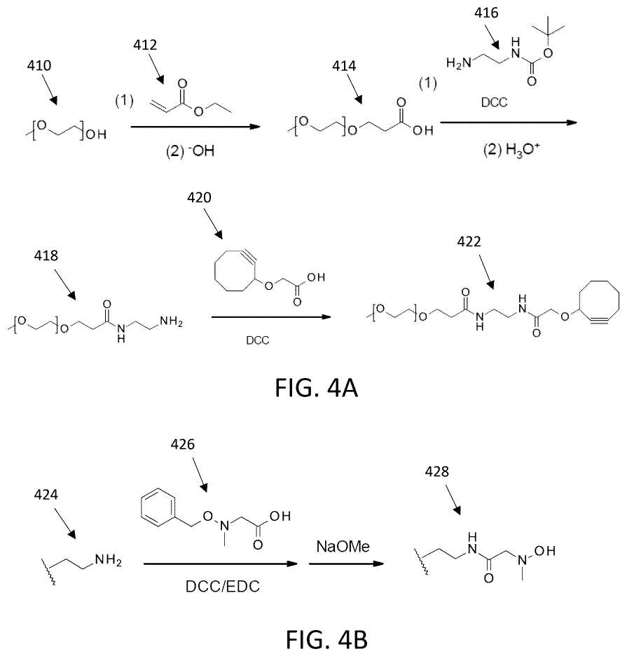

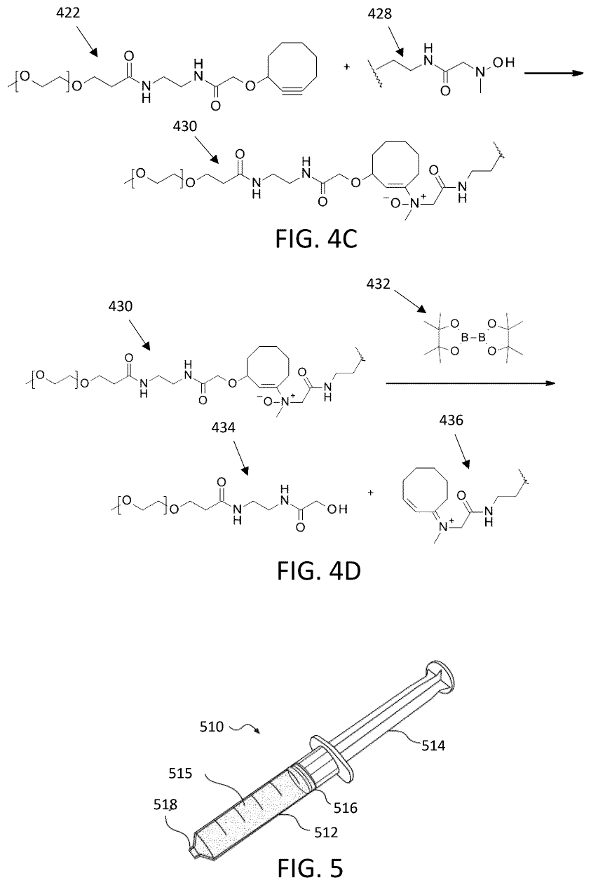

Turning to FIG. 4A, a hydroxyl-terminated multi-arm PEG (410) (a terminus of only one arm is illustrated) is reacted with acrylic acid ethyl ester (412) followed by treatment with base to yield a carboxyl-terminated multi-arm PEG (414). The carboxyl-terminated multi-arm PEG (414) is reacted with N-boc-1,2-diaminoethane (416), also known as tert-butyl N-(2-aminoethyl) carbamate, in the presence of a carbodiimide coupling agent such as DCC, followed by treatment in acid to form aminoethylaminocarbonyl-terminated multi-arm PEG (418), which is reacted with 2-(cyclooct-2-yn-1-yloxy) acetic acid (420) to form ethyl(cyclooct-2-yn-1-yloxy)aminocarbonyl-terminated multi-arm PEG (422) in which the multi-arm PEG is functionalized with a strained alkyne. Although a hydroxyl-terminated multi-arm PEG is employed in FIG. 4A, it will be appreciated that other hydroxyl terminated multi-arm polymers may be used including those containing the polymer chains described above.

With reference now to FIG. 4B, trilysine (424) (the terminus of only a single aminobutyl side chain out of the three aminobutyl side chain of the trilysine is shown) is reacted with N-Methyl-N-(phenylmethoxy)glycine (426) in the presence of a carbodiimide coupling agent such as DCC, followed by reaction sodium methoxide (NaOMe) to form an N-methylhydroxylamine-functional compound (428) in which N-methylhydroxylamine groups are linked to a trilysine residue through amide linkages. Although trilysine is used as a multi-functional amine in this specific instance, other multi-functional amines may be used, including those set forth above.

With reference to FIG. 4C, the cyclooctyne groups of the cyclooctyne-terminated multi-arm PEG (422) of FIG. 4A can be rapidly reacted with the N-methylhydroxylamine groups of the N-methylhydroxylamine-functionalized trilysine (428) of FIG. 4B to form a crosslinked hydrophilic polymer hydrogel (430) with crosslinks that contain enamine N-oxide groups, specifically, 1-(methyloxidoamino)cyclooctene groups.

Because the resulting hydrogel contains crosslinks that contain enamine N-oxide groups, which connect the multi-arm polymer residue and the trilysine-based crosslinker residue, when the hydrophilic polymer hydrogel is no longer desirable, it can be degraded on demand by treatment with a diboron compound. In a particular example shown in FIG. 4D, the crosslinked hydrophilic polymer hydrogel (430) of FIG. 4C is contacted with a diboron compound, specifically, bis(pinacolato)diboron (432), thereby breaking the crosslinks and forming a free N-(2-hydroxyethyl)amide-terminated arm (434), which is associated with a residue of the incorporated multi-arm PEG, and an imminium group (436), which is associated with a residue of the incorporated trilysine-based crosslinker.

In some embodiments, the present disclosure pertains to methods that comprise contacting a hydrogel filler material, which comprises hydrophilic polymer chains that are crosslinked by crosslinks that comprise immolative linkers or by crosslinks that contain reversible covalent linkages that incorporate reactive dimeric linkers, with a cleavage composition that comprises a singly reactive molecule. For example, in the event that it is desirable to remove a hydrophilic polymer hydrogel from a subject (e.g. because therapy is complete, because the crosslinked hydrophilic polymer is causing discomfort, etc.), the crosslinked hydrophilic polymer may be contacted with the cleavage composition.

In some embodiments, the cleavage composition contains a singly reactive molecule that acts to break the crosslinks at the position of the immolative linkers within the crosslinks. In some embodiments, the cleavage composition contains a singly reactive molecule that acts to break the crosslinks at the position of the dimeric linkers within the crosslinks.

In addition to one or more singly reactive molecules, the cleavage compositions of the present disclosure may contain one or more additional agents such as therapeutic agents, imaging agents, colorants, tonicity adjusting agents, suspension agents, wetting agents, and pH adjusting agents as detailed below.

In some aspects, the present disclosure provides hydrogel filler materials that comprise a reaction product of a multifunctional crosslinker and a multi-arm polymer having polymer chains that are reactive with the multifunctional crosslinker.

In some of these embodiments, the multifunctional crosslinker is a dimeric reactive linker, and hydrophilic polymer hydrogels are formed that comprise a reaction product of the dimeric reactive linker and a multi-arm polymer wherein functional groups at the ends of hydrophilic polymer chains that form the arms of the multi-arm polymer are reversibly crosslinked with the dimeric reactive linker.

In some of these embodiments, hydrophilic polymer hydrogels are formed that comprise a reaction product of the multifunctional crosslinker and a multi-arm polymer wherein functional groups of the multifunctional crosslinker irreversibly react with functional groups at the ends of hydrophilic polymer chains that form the arms of the multi-arm polymer, creating crosslinks that contain immolative linkers.

In some aspects of the present disclosure, systems are provided that are configured to deliver (a) a multifunctional crosslinker as described herein and (b) a multi-arm polymer as described herein. When the multifunctional crosslinker and the multi-arm polymer are comingled, crosslinks are formed between the multifunctional crosslinker and the multi-arm polymer. Such systems can be used to form hydrophilic polymer hydrogels in vivo within a subject, for example, using a delivery system like that shown in FIG. 2. Such systems can also be used to form polymer hydrogels ex vivo, which are subsequently introduced to a subject, for example, by providing particles of such hydrophilic polymer hydrogels which can be provided in a fluid suspension.

Such hydrophilic polymer hydrogels may be degraded by contact with a cleavage composition that is adapted to break down the crosslinks within the hydrophilic polymer hydrogels.

In some aspects, the present disclosure pertains to a system that comprises (a) an injectable or implantable composition, which comprises a pre-formed hydrophilic polymer hydrogel or which comprises precursors that can be combined to form a hydrophilic polymer hydrogel, and (b) a cleavage composition that acts to break the crosslinks within the hydrophilic polymer hydrogel.

Various cleavage compositions are described above and include those that contain at least one singly reactive molecule. The cleavage compositions may be provided in a suitable reservoir such as a syringe, vial or ampule. Whether supplied in a syringe, vial, ampule or other reservoir, the cleavage compositions may be provided, for example, in dry form (e.g., powder form) or in fluid form, such as in a solution form, or in a multi-phase fluid such as a suspension of particles of the at least one singly reactive molecule or in an oil/water or water/oil emulsion form, wherein singly reactive molecule is predominantly present in the oil phase or the aqueous phase. The cleavage compositions may further include one or more additional agents, including therapeutic agents, imaging agents, colorants, tonicity adjusting agents, suspension agents, wetting agents, and pH adjusting agents as described below.

FIG. 5 illustrates a syringe 510 for injection of a cleavage composition as discussed herein. The syringe 510 may comprise a barrel 512, a plunger 514, and one or more stoppers 516. The barrel 512 may include a Luer adapter (or other suitable adapter/connector) at the distal end 518 of the barrel 512, suitable for attachment to an injection needle or a flexible catheter. The syringe barrel 512 may serve as a reservoir, containing the cleavage composition 515 for injection into a subject, for example, through a needle or catheter.

When desired, cleavage of a hydrogel filler material can be initiated by contacting the hydrogel filler material with a cleavage composition as described herein.

In some embodiments the hydrogel filler material may comprise a hydrophilic polymer hydrogel that comprises crosslinks between hydrophilic polymer chains within the hydrophilic polymer hydrogels, which crosslinks contain immolative linkers or reversible covalent linkages that incorporate reactive dimeric linkers that are dispersed throughout the hydrogels, as described above.

As noted above, hydrogel filler materials for use in the present disclosure may contain one or more additional agents such as therapeutic agents, imaging agents, colorants, tonicity adjusting agents, and pH adjusting agents. In embodiments where the balloon permits release (e.g., by allowing diffusion through the balloon), such agents (e.g., therapeutic agents, imaging agents, etc.) may be released from the balloon over time.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, mRNA, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others. Examples of tonicity adjusting agents include sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others. Examples of pH adjusting agents including various buffer solutes.

In other aspects of the present disclosure, methods are provided that comprise: implanting a balloon in a subject at a target location and subsequently introducing a hydrogel filler material into the balloon. In some embodiments, the balloon is implanted with the assistance of a delivery sheath. For example, the delivery sheath can first be inserted into the subject. Then, the balloon can be inserted through the sheath to the target location, after which the hydrogel filler material is introduced into the balloon. As another example, the balloon may be disposed within the delivery sheath while the delivery sheath is inserted into the subject. Then, the balloon is advanced from the delivery sheath to the target location and filled with the hydrogel filler material. In some embodiments, the balloon is implanted without the assistance of a delivery sheath.

In various embodiments, the hydrogel filler material is introduced into the balloon through an elongate filling tube, or catheter, which is removably coupled to the balloon and is in fluid communication with an interior of the balloon. After filling the balloon, the filling tube is detached from the balloon and withdrawn from the subject, along with the delivery sheath, if employed. As described above, in some embodiments, outflow of the hydrogel filler material from the balloon can be prevented, for example, through the use of a check valve, a resealable aperture, or a plug. In some embodiments, the plug is located inside the balloon and is detachably connected to a distal end of the filling tube, such that when the filling tube is withdrawn, the plug is fitted within the aperture inside the balloon. In some embodiments, outflow of the hydrogel filler material from the balloon is prevented through the use of a shear-thinning filler material or by forming a hydrogel filler material in situ in the balloon.

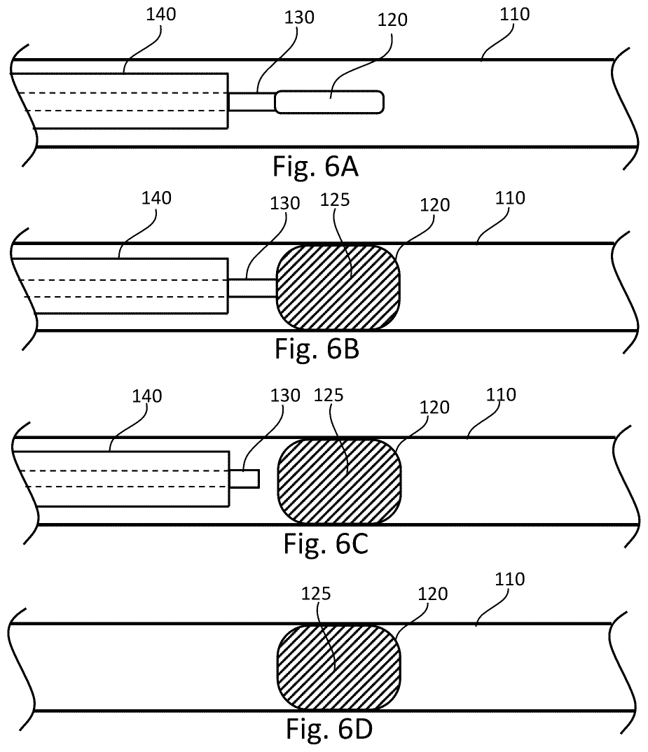

In a particular embodiment shown in FIGS. 6A-6E, a balloon 120 is inserted at a target site in body lumen, such as a blood vessel 110, while in substantially uninflated state. The balloon 120 is removably coupled to a distal end of a filling tube 130 (e.g., a catheter), which has a lumen that is in fluid communication with an interior of balloon 120. In some embodiments, the balloon may be rolled or folded for this purpose. In the embodiment shown, the balloon is inserted with the assistance of a delivery sheath 140. As shown in FIG. 6B, the filling tube 130 is then used to inflate the balloon 120 with a hydrogel filling material 125, such that the balloon 120 occludes the blood vessel 110. The filling tube 130 is then decoupled from the balloon 120 as shown in FIG. 6C. Then, the filling tube 130 and delivery sheath 140 are removed from the subject as shown in FIG. 6D, completing the implantation procedure.

In some embodiments, the hydrogel filling material 125 of the present disclosure can be imaged during or after administration using a suitable imaging technique such as ultrasound, or an X-ray-based imaging technique, such as computerized tomography or X-ray fluoroscopy.

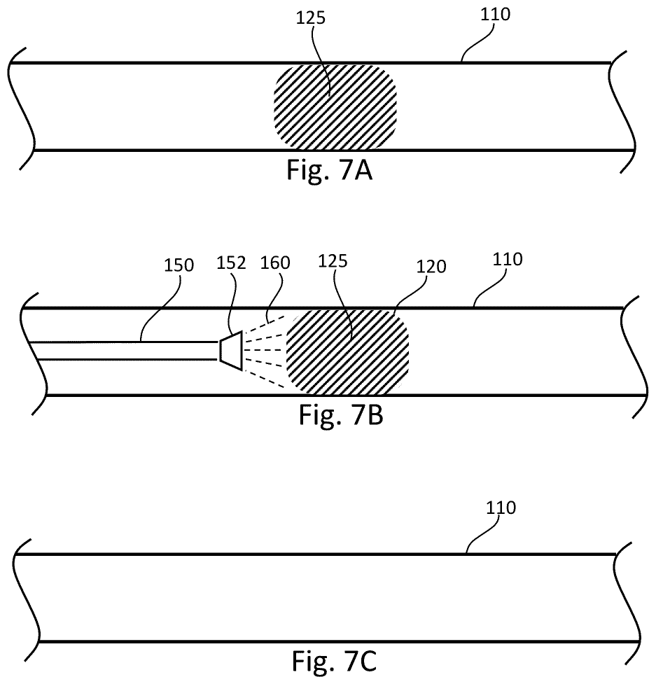

As previously noted, in some embodiments, a degradable balloon material is selected. Degradation may occur due to various mechanisms that include hydrolysis by interaction with water in the body, enzymatic degradation by enzymes that are naturally occurring in the body or enzymes that are introduced into the body, oxidation by oxidants produced by the body, and/or by breakdown using cleavage compositions, among other possible mechanisms. Regardless of the mechanism, after the polymeric material forming the balloon degrades, the hydrogel filling material 125, which may be a biostable or a biodegradable solid, may remain in place, at least for a time, as shown in FIG. 7A.

In some embodiments, cleavage of the hydrogel filling material 125 can be initiated by contacting the hydrophilic polymer hydrogel compositions with a cleavage composition as described above.

Turning again to FIG. 7A, the hydrogel filling material 125 may be formed from a polymeric material comprising hydrophilic polymer chains that are linked by crosslinks that contain immolative linkers or reversible covalent linkages that incorporate reactive dimeric linkers. When degradation of the hydrogel filling material 125 is desired, a catheter 150 having a distribution head 152 may be advanced to the hydrogel filling material 125 as shown in FIG. 7B, and a cleavage composition 160 may distributed from the distribution head 152 onto the hydrogel filling material 125. When the cleavage composition 160 contacts the hydrogel filling material 125, the immolative linkers or the reversible covalent linkages that incorporate reactive dimeric linkers are cleaved, resulting in degradation of the hydrogel filling material 125 leaving the blood vessel 110 non-occluded as shown in FIG. 7C.

In other embodiments, the hydrogel filling material 125 is a biodegradable solid, and the hydrogel filling material 125 ultimately biodegrades from natural processes (e.g., hydrolysis, enzymatic degradation, oxidation, etc.) leaving the blood vessel 110 non-occluded as shown in FIG. 7C.

In other aspects, the present disclosure provides balloon implantation kits that include one or more inflatable balloons configured to be implanted in a human body and one or more containers that include a filler material that is configured to be introduced into the one or more balloons or that include filler material precursors that, when combined, produce a filler material in the one or more balloons. Containers for the filler material or the filler material precursors include, for example, vials, ampules, empty syringes, and preloaded syringes. In some embodiments, the one or more inflatable balloons further include features for preventing outflow of filler material such as check valves, plugs or constricting devices as described above.

In some embodiments, the balloon implantation kits include one or more filling tubes (e.g., catheter tubes) for introducing the filler material or filler material precursors into the one or more balloons. In some embodiments, the one or more filling tubes are releasably attached to the one or more balloons.

In some embodiments, the balloon implantation kits include one or more delivery sheaths through which the one or more balloons is/are implanted in the subject. In some of these embodiments, the one or more balloons is/are prepackaged in the one or more delivery sheaths.

In some embodiments, the balloon implantation kits include one or more containers that contain a cleavage solution as described herein. Containers for the cleavage solution include, for example, vials, ampules, and preloaded syringes. In these embodiments, the balloon implantation kits may further include an elongated tube (e.g., a catheter tube) for delivering the cleavage solution. In some these embodiments, a distribution head, such as a spray head, may be disposed at a distal end of the catheter tube.

The balloon implantation kits may further include sterile packaging in which the above-described the kit components are removably packaged in a sterile state.

1. A balloon implantation kit comprising: (a) a balloon configured to be implanted in a mammalian body and (b) a reservoir containing a filler material that is configured to be injected into the balloon, the filler material selected from a hydrogel filler material comprising crosslinked hydrophilic polymer chains or hydrogel precursors that form a hydrogel filler material in the balloon when combined.

2. The balloon implantation kit of claim 1, wherein the crosslinked hydrophilic polymer chains are crosslinked by crosslinks that comprise hydrolysable linkers.

3. The balloon implantation kit of claim 2, wherein the hydrolysable linkers are selected from a carbonate linker, an acid anhydride linker, an imide linker, a ketal linker, a carbamate linker, an organophosphate ester linker, a silane linker, an amide linker, a hydrozonium linker, an acylhydrozone linker, an oxime linker and an amidohydrozone linker.

4. The balloon implantation kit of claim 2, wherein the crosslinks further comprise an activating group positioned adjacent to the hydrolysable linkers, which increases a rate of hydrolysis of the hydrolysable linkers.

5. The balloon implantation kit of claim 4, wherein the activating group is selected from a hydrogen bond donor, a hydrogen bond acceptor, a lone pair donor, a lone pair acceptor, a silicon containing group, a boron containing group, a phosphonate group and a sulfonate group.

6. The balloon implantation kit of claim 4, wherein the activating group is selected from a urea linker, a urea pendant group, a thiourea linker, a thiourea pendant group, an amine linker, an amine pendant group, an alcohol pendant group, a silicon-containing linker, a silicon-containing pendant group, a boron-containing linker, a boron-containing pendant group, a phosphonate linker, a phosphonate pendant group, and a sulfonate pendant group.

7. The balloon implantation kit of claim 1, wherein the crosslinked hydrophilic polymer chains are crosslinked by crosslinks that comprise immolative linkers or by crosslinks that comprise reversible covalent linkers that incorporate reactive dimeric linkers.

8. The balloon implantation kit of claim 7, wherein the kit further comprises a cleavage composition that contains a singly reactive molecule that acts to break the crosslinks at a position of the immolative linkers within the crosslinks or wherein the cleavage composition contains a singly reactive molecule that acts to break the crosslinks at a position of the dimeric linkers within the crosslinks.

9. The balloon implantation kit of claim 8, wherein the kit comprises a syringe barrel that contains the cleavage composition.

10. The balloon implantation kit of claim 8, wherein the hydrophilic polymer hydrogel comprises the hydrophilic polymer chains that are crosslinked by crosslinks that comprise immolative linkers and wherein the hydrogel cleavage composition contains the singly reactive molecule that acts to break the crosslinks at the position of the immolative linkers within the crosslinks.

11. The balloon implantation kit of claim 8, wherein the hydrophilic polymer hydrogel comprises the hydrophilic polymer chains that are crosslinked by crosslinks that contain reversible covalent linkers that incorporate a reactive dimeric linker and wherein the hydrogel cleavage composition contains the singly reactive molecule that acts to break the crosslinks at the position of the dimeric linkers within the crosslinks.

12. The balloon implantation kit of claim 1, wherein the hydrophilic polymer chains are selected from polyalkylene oxide chains, polyester chains, polyoxazoline chains, polydioxanone chains, polypeptide chains, polyacrylate chains, and polyacrylamide chains, or combinations thereof.

13. The balloon implantation kit of claim 1, wherein the filler material is a shear-thinning hydrogel material.

14. The balloon implantation kit of claim 13, wherein the shear-thinning hydrogel material comprises fibrous protein, silicate particles and water.

15. The balloon implantation kit of claim 1, wherein the filler material is a lower critical solution temperature material that is in the form of a hydrogel at body temperature.

16. The balloon implantation kit of claim 1, wherein the filler material comprises hydrogel precursors that form a hydrogel in the balloon when combined.

17. The balloon implantation kit of claim 1, wherein balloon is biodegradable.

18. A balloon implantation kit comprising: (a) a balloon configured to be implanted in a mammalian body; (b) a reservoir containing a filler material that is configured to be injected into the balloon, the filler material selected from a hydrogel filler material comprising crosslinked hydrophilic polymer chains or hydrogel precursors that form a hydrogel filler material in the balloon when combined; and (c) an elongate filling tube that is configured to be removably coupled to the conformable, fillable balloon, the elongate filling tube having a lumen that is configured for fluid communication with an interior of the conformable, fillable balloon.

19. The balloon implantation kit of claim 18, wherein the kit further comprises a delivery sheath.

20. A method comprising contacting an implant, which implant comprises a conformable, fillable balloon and a hydrogel filler material disposed within the conformable, fillable balloon that comprises a hydrophilic polymer hydrogel comprising crosslinked hydrophilic polymer chains that are crosslinked by crosslinks that comprise immolative linkers or by crosslinks that contain reversible covalent linkages that incorporate reactive dimeric linkers, with a cleavage composition that contains a singly reactive molecule that acts to break the crosslinks at a position of the immolative linkers within the crosslinks or at a position of the dimeric linkers within the crosslinks, thereby accelerating breakdown of the conformable, fillable balloon.
